(19) United States
(12) Patent Application Publication
(10) Pub. No.: US 2008/0058652 A1
(43) Pub. Date: Mar. 6, 2008

(54) Medical Devices

(76) Inventor: Peter Alfred Payne, Devon (GB)

Correspondence Address: James E. Bradley;BRACEWELL & GIULIANI, P.O.Box 61389, Houston, TX, 77208-1389, US

(21) Appl. No.: 11/666,948

(22) PCT Filed: Nov. 4, 2005
(86) PCT No.: PCT/GB05/04265

§ 371 Date: Oct. 2, 2007

(30) Foreign Application Priority Data
Nov. 4, 2004 (GB) 2004/004661
Dec. 21, 2004 (GB) 0428000.4

(51) Int. Cl. A61B 5/0215

(52) U.S. Cl. 600/488; 623/1.1; 623/2.1

(57) ABSTRACT

There is provided a medical device for implantation in a body including: a substrate formed from a material capable of acting as an active sensing material for sensing a physiologically or clinically relevant parameter; at least one sensor for sensing a physiologically or clinically relevant parameter, each sensor including a portion of the substrate which is configured to act as an active sensing material; and telemetric communication means coupled to said at least one sensor for telemetrically transmitting data related to a parameter sensed by the at least one sensor to a remote device.

This invention relates to implantable medical devices, with particular, but by no means exclusive, reference to heart valve devices and implants for same.

Implantable devices which transmit telemetric data relating to the patient in which the device is implanted are known. US 2002/0072656 A1 and U.S. Pat. No. 6,409,675 (the contents of which are herein incorporated by reference) disclose apparatus which are implanted into the vascular system of an individual and which are capable of providing information relating to clinically important parameters. Further prior art relating to the collection of in vivo data in a body comprises U.S. Pat. Nos. 6,667,725, 6,486,588, 6,729,336, 6,645,143, 6,658,300, 5,967,986, 6,743,180 and 6,592,518; US Patent Application 2003/0136417; International Patent Publications WO 03/061467, WO 03/061504 and WO 04/014456, and our co-pending International Patent Application PCT/GB2004/004661, the contents of all of which are herein incorporated by reference.

The present invention, in at least some of its embodiments, provides improved implantable medical devices which are easy to manufacture, economical to produce, more efficient, and readily provided in a range of clinically useful designs.

For the avoidance of doubt, the terms “patient” and “body” as used herein includes both humans and animals within its scope.

According to a first aspect of the invention there is provided a medical device for implantation in a body including:

a substrate formed from a material capable of acting as an active sensing material for sensing a physiologically or clinically relevant parameter;

at least one sensor for sensing a physiologically or clinically relevant parameter, each sensor including a portion of the substrate which is configured to act as an active sensing material; and

telemetric communication means coupled to said at least one sensor for telemetrically transmitting data related to a parameter sensed by the at least one sensor to a remote device.

The telemetric communication means may include at least one antenna.

Preferably, the telemetric communication means is disposed, at least in part, on the substrate. In some embodiments the telemetric communication means is wholly disposed on the substrate.

In a preferred embodiment, the antenna is disposed on the substrate.

In an alternative embodiment, the device further includes an additional substrate having the antenna formed thereon, the additional substrate contacting the substrate formed from a material capable of acting as an active sensing material.

Preferably, the substrate is substantially planar or bent from a substantially planar configuration. These configurations are particularly useful for in vivo applications.

In preferred embodiments, one face of the substrate has an earth plane disposed thereon. Advantageously, the at least one sensor is disposed on a front face of the substrate, and the earth plane is disposed on a back face of the substrate so as to extend at least over a region which is in register with the area defined by the at least one antenna, and, preferably, to extend additionally over a region which is in register with the area defined a sensor. This configuration can provide advantages both in the construction of and operation of the sensor and in the efficiency of the telemetric data communication. Conveniently, the earth plane is disposed over substantially the entire back face of the substrate.

According to a preferred aspect of the invention the medical device is adapted to be implanted in the heart of a patient and operable therein i) as a heart valve; or ii) to assist in the functioning of one of the patient's heart valves; or iii) to monitor the functioning of one of the patient's heart valves:

In the case of option i), above, the medical device is a heart valve which may further comprise a valve for regulating the flow of blood through the device. Typically, the valve comprises a number of leaflets, although this is not a limiting feature of the invention.

In the case of option ii), above, the medical device may comprise a heart valve repair device. The heart valve repair device may comprise a heart valve support structure, such as an annular support structure. Such annular structures may be sewn onto a patient's dysfunctional heart valve.

In the case of option iii), above, the medical device may comprise a structure suitable for placement in or on a patient's heart valve. The patient's heart valve may be a treated indigenous valve or a valve which, although untreated, might require monitoring to determine when or if future treatment or replacement is required.

The medical device may be a tissue valve device having a valve wall formed from tissue. The medical device may be stented or stentless. In particular, the medical device may further comprise a stent support for the valve wall, in which at least one sensor and the telemetric communication means are disposed between the stent support and the valve wall.

A tissue valve medical device may further comprise a protective cover disposed around the periphery of the device, and the at least one sensor and the telemetric communication means may be disposed between the valve wall and the protective cover. The protective cover may comprise a polymeric layer, such as Dacron (RTM) or a pericardial layer, typically one that has been crosslinked.

The medical device may be a mechanical heart valve.

Alternatively, the medical device may be a graft or a stent, or attached to a graft or a stent.

The device may be attached to a desired body part by suitable means, such as by suture or adhesive. In preferred embodiments, the medical device is in a form suitable for injection into a body. The term “a form suitable for injection into a body” means that the device is at least capable of being introduced into a body in a clinically acceptable manner, for example, via an endoscope. It may be possible to provide a medical device suitable for injection using smaller injection means, such as a hypodermic needle.

In further embodiments of the invention, the medical device is an artificial heart or a left ventricular support device (LVad). Such devices contain artificial heart valves. It is possible for the substrate, sensor(s) and telemetric communication means to be disposed on the artificial heart valves, or, alternatively, the substrate, sensor(s) and telemetric communication means may be disposed elsewhere within or on the device.

In preferred embodiments, the telemetric communication means is a passive device. For example, the device may be powered by energy transmitted by a remote device.

The telemetric communication means may be a transponder, such as an RF tag device, also known as a Radio Frequency Identification (RFID) device. Such devices are extremely economical to utilise. Also, such devices can conveniently provide useful information, for example a record of the original performance data of the device, the device type, the location at which the device was inserted, details of the procedure, etc.

The medical device may further include a capacitor and means for receiving an externally applied source of energy and charging said capacitor using the externally applied source of energy, in which the telemetric communication means and/or a sensor is powered by charge stored in the capacitor. For example, an external radio frequency source can be used to charge a capacitor which would gradually discharge and power up the telemetric communication means and/or a sensor. The commencement of the powering of the telemetric communication means and/or a sensor by the capacitor can be controlled by control means. The control means causes the capacitor to be discharged at desired junctures, which may be at pre-determined times, or when the control means receives a control signal delivered from a device external to the body, such as an appropriate radio frequency signal.

Alternatively, the telemetric communication means may be powered by an energy source disposed on or in physical connection with the medical device, such as a battery.

Alternatively still, it may be possible to utilise energy produced by the patient, in particular energy associated with the beating of a patient's heart or other bodily functions which alter pressure to power the telemetric communication means. In such embodiments, the medical device may further comprise a capacitor and means for charging said capacitor using energy associated with a physiological event, in which the telemetric communication means and/or a sensor is powered by charge stored on a capacitor. Preferably, the means for charging said capacitor comprises a piezoelectric device, such as a polyvinylidene fluoride (PVDF) piezoelectric device. The piezoelectric device can produce the necessary electrical charge by transduction of the pulsating pressure changes inherent in blood pumped by the heart. The discharging of the capacitor to power the telemetric communication means and/or a sensor may be controlled by control means. The control means causes the capacitor to be discharged at desired junctures, which may be at pre-determined times, or when the control means receives a control signal delivered from a device external to the body, such as an appropriate radio frequency signal. It is possible for the means for charging said capacitor using energy associated with a physiological event to also act as a sensor. Piezoelectric sensors are particularly useful in this regard.

The telemetric communication means may be powered by an RF field.

The telemetric communication means may transmit data using an RF field.

The telemetric communication means may transmit data by other means and/or be powered by other means, such as microwave or other electromagnetic radiation, acoustic signals or other electromagnetic fields.

In further embodiments, the telemetric communication means, the means by which the telemetric communication means transmits data and the means by which the telemetric communication means is powered may utilise technology known in the field of mobile telephones (also known as cell telephones). In such embodiments, the telemetric communication means may transmit data using Bluetooth (RTM), WLAN, GSM, GPRS or UMTS technology.

The telemetric communications means may include an integrated circuit.

The integrated circuit may be disposed on the substrate or, alternatively, on the additional substrate.

The telemetric communication means may include a chip, preferably a microchip.

At least one sensor may be a pressure sensor for sensing blood pressure. In this way, highly relevant clinical pressure data, such as systolic and diastolic pressures, and pressure profiles as a function of time, can be obtained. Additionally, leakage can be detected by detecting changes in pressure. Leakage from vascular implants such as vascular grafts can be advantageously detected in this manner. Advantageously, the medical device comprises at least two spaced apart pressure sensors for sensing blood pressure at different locations, such as different locations in the heart of the patient. In this instance the telemetric communication means may telemetrically transmit data related to the difference in the blood pressures sensed by the at least two pressure sensors. In this way, information on blood flow and blood leakage can be obtained, particularly pressure differences across a valve or valve replacement, giving valuable data concerning valve narrowing/stenosis/incompetence. Pressure data including instantaneous pressure data can be obtained. Additionally, instantaneous blood velocity can be calculated.

At least one sensor may be an acoustic sensor for sensing acoustic signals. In this way, highly relevant clinical data relating to heart beat can be obtained. In particular the performance of heart valve(s) repair may be assessed, taking into consideration any abnormal rhythm and thus pressure profiles that might affect the interpretation of the telemetrically produced acoustic signal of the valve(s) performance. Additionally, information relating to blood flow, eg, whether blood flow is normal or abnormal, can be obtained.

Advantageously, the one or more sensors comprise at least one pressure sensor and at least one acoustic sensor for sensing blood pressure and acoustic signals. Blood pressure, pressure profiles and pressure differences may be sensed. A single sensor may sense blood pressure and acoustic signals.

One or more sensors may sense other physiologically relevant parameters, such as temperature, pH, biochemical parameters, CO₂ and O₂.

At least one sensor may be a passive sensor, ie, a sensor that does not require a power source in order to operate as a sensor.

The at least one sensor may be a piezoelectric sensor. The substrate may be formed from a polymeric material capable of acting as an active piezoelectric sensing material. The polymeric material may comprise polyvinylidene fluoride (PVDF) or a related PVDF material. PVDF is a preferred material since it is possible to provide PVDF sensors that can monitor both pressure and acoustic signals. Related PVDF materials include copolymers with PVDF, such as a PVDF-trifluorethylene (TrFe) copolymer. Other materials capable as acting as an active piezoelectric sensing material include ceramics and ceramic/polymer mixtures.

It is preferred that the substrate is formed from a deformable material, such as a polymer or a ceramic/polymer mixture, because in practical, in vivo applications placement of the device is much easier if the device can bend.

At least a portion of the medical device may be coated with a non-thrombogenic or anti-thrombogenic, bio-compatible substance. In particular, the one or more sensors may be coated in this manner.

The one or more sensors and telemetric communication means may be sealed within a bio-compatible protective structure. The protective structure may be coated with a non-thrombogenic or anti-thrombogenic bio-compatible substance.

According to a second aspect of the invention there is provided a medical device for implantation in a body including:

a substrate or a substrate stack including a plurality of stacked substrates, the substrate or substrate stack having opposed first and second faces;

at least one sensor disposed on the substrate or substrate stack for sensing a physiologically or clinically relevant parameter;

telemetric communication means, coupled to the at least one sensor, for telemetrically transmitting data related to a parameter sensed by the at least one sensor to a remote device, the telemetric communication means including at least one antenna disposed on the first face of the substrate or substrate stack; and

an earth plane disposed on the second face of the substrate or substrate stack and extending at least over a region which is in register with the area defined by the at least one antenna.

Typically, the telemetric communication means is disposed, either wholly or in part, on the substrate or substrate stack. The second aspect of the invention may incorporate features of the first aspect of the invention.

According to a third aspect of the invention there is provided a system for monitoring a patient including:

a medical device according to the first, second or fifth aspects of the invention; and

a remote device for receiving data telemetrically transmitted by the telemetric communication means.

In an important aspect, the invention provides a system for monitoring a patient including a medical device according to the second aspect of the invention and a remote device for receiving data telemetrically transmitted by the telemetric communication means; in which the remote device includes at least one data receiving antenna and an earth plane extending at least over a region which is in register with the area defined by the at least one data receiving antenna so as to improve the reception of data transmitted telemetrically by the medical device.

The remote device may be adapted to provide power remotely to the telemetric communication means. The remote device may be adapted to produce an RF field for this purpose.

The present invention includes within its scope the provision of one or more intermediate relay devices. In such embodiments, a relay device receives data transmitted by the telemetric communication means and sends the data on to the remote device or another relay device. A relay device may be implanted at a suitable position in the body. Relay devices are of particular importance if the telemetric communication means possesses only a short range.

Devices of the invention may be used in the heart, other organs, vessels, lungs, orthopaedic uses, neurosurgical applications, the urinary system, the digestive system, intensive care monitoring and with transplanted organs such as the kidneys, liver, or heart, for example, to assess flow and pressure differentials on in-flow and out-flow vessels as a guide to rejection or response to therapy. A non-limiting embodiment comprises the use of medical devices in the treatment and/or monitoring of an Abdominal Aortic Aneurysm (AAA), in particular in the instance in which the medical device is an AAA graft. Vessels might be arteries, such as the pulmonary artery and the aorta, and veins. Use in intensive care monitoring includes the placement of medical devices of the invention on vessels, such as the pulmonary artery and the aorta in one or more positions, for example, to measure cardiac output and filling pressures at or after open operations such as heart and lung operations. The device may also be used in non-surgical cases where patients have life threatening conditions such as trauma, heart failure or septicaemia. Orthopaedic use includes use on ligaments and joints, for example onto joint capsules or into joint replacement such as hips and knees where stress/strain is an important additional measurement. Neurosurgical use includes measurement of intracranial pressure and intraspinal pressures. Devices of the invention might be placed on the membrane of the brain (dura). Use relating to the lung includes the detection of airway obstruction, particularly in asthma cases, and also measuring the response of asthma cases and other lung conditions to medications. Lung conditions such as emphysema, chronic bronchitis, and other forms of restrictive airway disease might be monitored. Urinary system use includes measurement of bladder pressure, ureteric flow or back flow, and urinary flow. Further uses relate to valve repair and carotic patches following endarterectomy. In the latter instance carotic flows can be measured, and the device can be sewn in place. ECG measurements might be made. Other preferred uses of the invention include uses relating to stents and vascular grafts. Vascular grafts may be with or without stents. The medical devices of the invention may themselves comprise the stent or vascular graft itself, or, alternatively, the medical device may be disposed on, in, or in the vicinity of the stent or vascular graft. The medical device may be used to monitor the performance of the stent or vascular graft, for example, by detecting whether leakage or stenosis is occurring, or to assess whether the stent or vascular graft has moved. Non-limiting examples of stents include pulmonary, vascular, coronary, thoracic and abdominal stents. Non-limiting examples of grafts include AAA, infrainguinal, femoral popliteal, femoral distal, vein and vascular access grafts. Examples of vascular access grafts include grafts for patients requiring dialysis and paediatric cardiac conduits. Digestive system measurements may be made, for example, pH and pressure, particularly in the oesophagus, stomach and bowel.

According to a fourth aspect of the invention there is provided a sensor including a material capable of acting as an active sensing material and an electrode formed on the material in an antenna pattern so as to i) enable the material to be operated as an active sensing material and ii) act as an antenna to transmit and/or receive signals relevant to the operation of the sensor.

Preferably, the material is a material capable of acting as an active piezoelectric material. Advantageously, the material is polymeric material, preferably PVDF.

The antenna pattern (which is typically a spiral shape) will leave one or more gaps between adjacent portions of the electrode, and it is highly preferred that the gaps are minimised so that the antenna covers as much of the active area of the material as possible. Generally, it is desirable that the gaps are of less than 50 μm, preferably less than 25 μm, most preferably about 10 μm.

Advantageously, the sensor further includes an earth plane formed on the material and extending at least over a region of the material which is in register with the area defined by the electrode.

The sensor can be used in numerous applications. In a preferred application, the sensor is in the form of a medical device suitable for implantation in a body, in which the sensor is for sensing a physiologically or clinical relevant parameter, and the electrode telemetrically transmits the data related to a parameter sensed by the sensor to a remote device.

The sensor may incorporate features of the first and or second aspects of the invention.

According to a fifth aspect of the invention there is provided a medical device for implantation in a body including:

at least two sensors for sensing a physiologically or clinically relevant parameter;

differential measurement means for measuring differences in the responses of the sensors; and

telemetric communication means coupled to the differential measurement means for telemetrically transmitting data related to the differences in the responses of the sensors to a remote device.

The differential measurement means may include a bridge arrangement, such as a Wheatstone bridge.

The differential measurement means may include a differential amplifier.

In this way, differential measurements may be directly transmitted by the device. In preferred embodiments, differential pressure and/or acoustic measurements can be transmitted so that, for example, pressure and/or acoustic differentials across the leaflets of a heart valve might be measured. Additionally or alternatively, the effect of interferences to the sensors' responses can be compensated for.

The device of the fifth aspect of the invention may incorporate features of the first, second or fourth aspects of the invention.

Embodiments of medical devices and systems in accordance with the invention will now be described with reference to the accompanying drawings, in which:

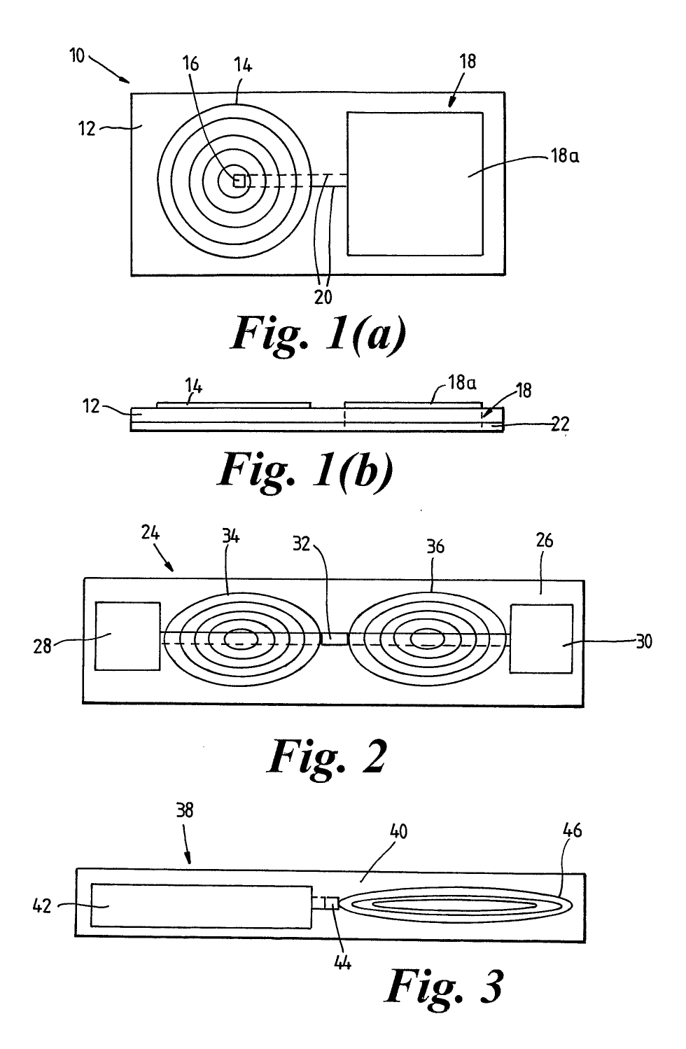

FIG. 1 shows (a) a plan view and (b) a side view of a first embodiment of a medical device;

FIG. 2 shows a plan view of a second embodiment of a medical device;

FIG. 3 shows a plan view of a third embodiment of a medical device;

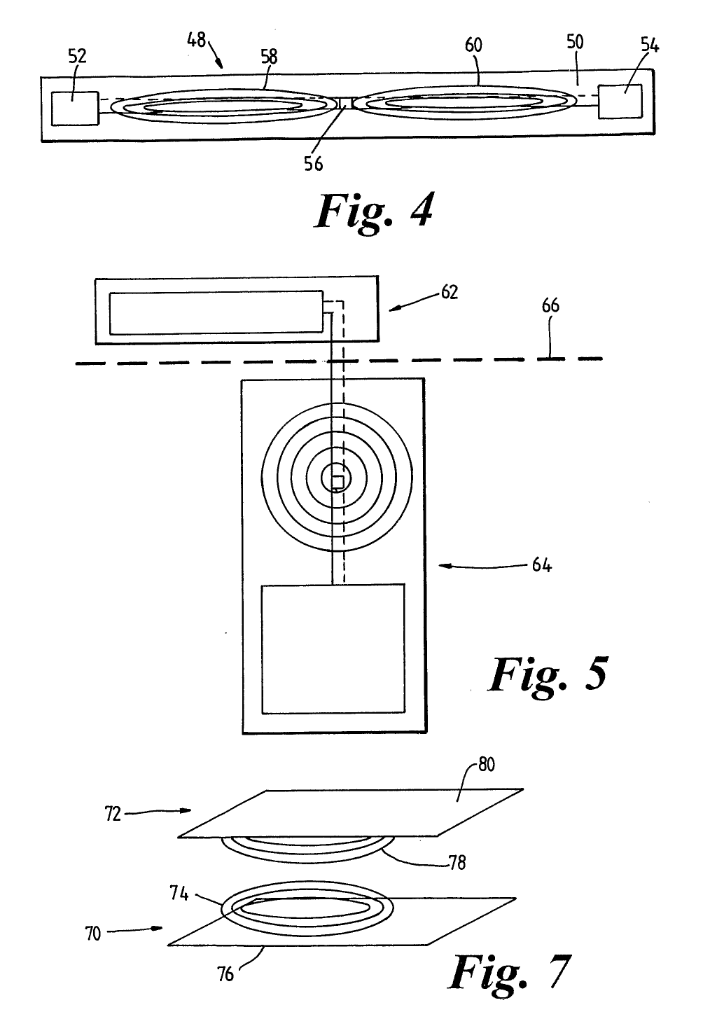

FIG. 4 shows a plan view of a fourth embodiment of a medical device;

FIG. 5 shows an arrangement for making differential measurements across heart valve leaflets;

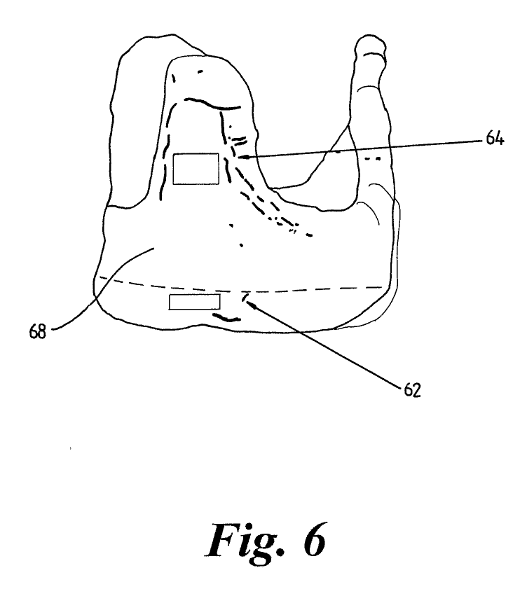

FIG. 6 shows the placement of two devices across an implantable tissue heart valve;

FIG. 7 shows an arrangement using earth planes behind an implantable medical device and a reader device;

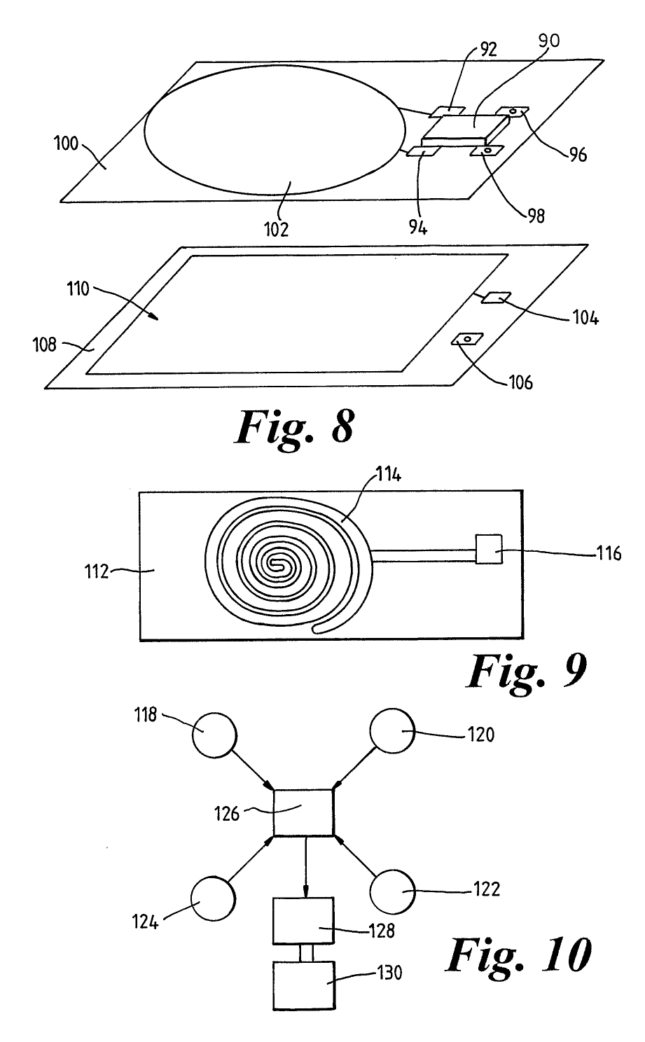

FIG. 8 shows an exploded view of a fifth embodiment of a medical device;

FIG. 9 shows a plan view of a sixth embodiment of a medical device;

FIG. 10 shows a differential sensor measurement arrangement.

FIGS. 1 to 5 show various embodiments of medical devices in accordance with the invention. In each of these Figures, antennae are shown semi schematically in the form of concentric circles and ovals. It should be noted that these concentric circles and ovals are intended to denote the spiral geometry required for real antennas. FIG. 1 depicts a first embodiment of a medical device, shown generally at 10, comprising a substrate 12 of a material which is capable of piezoelectric activity. The substrate 12 can be of any convenient shape and dimensions. On a first face of the substrate 12 are disposed telemetric communication means 14, 16 and a piezoelectric sensor 18. The telemetric communication means comprises an integrated circuit 16 which is in communication with an antenna 14. The integrated circuit 16 also has interconnections 20, such as gold wiring, to the piezoelectric sensor 18. If required, further circuitry, such as a buffer amplifier and digital to analogue converter, together with associated interconnections, can be carried on the substrate 12 as well. The piezoelectric sensor 18 is formed by providing an electrode 18a over a subset of the substrate 12, for example by metallisation. Only the region of the substrate 12 underneath the electrode 18a acts a piezoelectric sensor, which can be used, for example, for acoustic and/or pressure sensing. All of the other regions of the substrate 12 are essentially piezoelectrically inert. On the opposite face to the face of the substrate 12 shown in FIG. 1(a) is an earth plane 22. The earth plane 22 may be formed by depositing a metallic material such as gold over the surface of the substrate 12. It is preferred that the earth plane is disposed over the entire face of the substrate 12, although it is not necessary that this is the case. Rather, the earth plane could be formed in one or more specific regions of the substrate 12. The earth plane 22 extends underneath the piezoelectric sensor 18, and it is noted that only the portion of the substrate 12 underneath the electrode 18a on the top surface of the substrate 12 is electrically poled, making it an active piezoelectric material. In addition to assisting in the operation of the piezoelectric sensor, the earth plane 22 performs an additional role by improving the operation of the antenna 14. Interconnections between the front and back faces of the substrate 12, for example from the earth plane 22 to the integrated circuit 16, can be made by plating through apertures in the substrate 12, or through the use of miniature rivets.

It is preferred that the substrate 12 is formed from PVDF. An advantage of PVDF (and other polymeric piezoelectric materials) is that is formable to some extent, and thus the substrate can undergo some bending. This is highly advantageous for use in vivo applications. An advantage with PVDF piezoelectric sensors is that they can be operated both as a pressure transducer and as a microphone, monitoring acoustic signals. In a pressure transducer mode, the PVDF sensor might react to blood pressure during the heart cycle. In the microphone mode, the PVDF transducer might listen to the sounds emitted by the blood as it moves through a heart valve. Details of the use of PVDF as a pressure sensor and as a microphone can be found in “Tactile Sensors for Robotics in Medicine”, edited by John G Webster, John Wyley, 1988, particularly chapter 8 “Piezoelectrics sensors”, and “The Applications of Ferroelectric Polymers”, Chapman and Hall, 1988, in particular chapter 8, “Microphones, Headphones and Tone Generators”, the contents of both of which are herein incorporated by reference. Representative thicknesses for the substrate are between 60 and 150 μm, preferably between 60 and 110 μm. In preferred embodiments, the integrated circuit 16 is a so called RF tag device (such devices are also known as radio frequency identification (RFID) chips—see, for example, UK Periodical “Computing”, 16 Jan. 2003 edition). Such devices are well known for position monitoring purposes. For example, animals such as cattle and pets may be monitored in this way using an RF tag positioned subcutaneously. RF tag devices are passive devices until interrogated by a suitable, and typically relatively powerful, RF signal. The signal is energetic enough to power up the RF tag device which, in the context of position measurement, typically responds with some form of electronic bar code signal, typically using a response frequency around 450 MHz. For the purposes of the present invention, the function of the RF tag is altered somewhat from these prior art applications. In particular, the RF tag accepts data from the sensor, and transmits data relating to measurements made by the sensor the interrogating remote device. One way in which this can be achieved is to use the signal from the sensor to modulate the response from the RF tag in a suitable manner. A preferred way in which data is transmitted by the RF tag is by modification of the ID code of the RF tag in response to the data accepted from the sensor. Other methods for transmitting data, such as modulating the data transmission rate to the RF tag, might be contemplated. Suitable powering and data collection regimes would suggest themselves to the skilled person. Further information concerning the operation of RF tags can be found in International Patent Publication WO 02/073523 and U.S. Pat. No. 6,622,567, the contents of both of which are herein incorporated by reference.

The first embodiment shown in FIG. 1 can be used in a number of applications, such as in heart valves. The device 10 could be accommodated on stented, semi-stented, or stentless heart valves, for example.

FIG. 2 shows a second embodiment of a medical device, depicted generally at 24. The device 24 comprises a substrate 26 having formed therefrom two sensors 28, 30, and telemetric communication means comprising an integrated circuit 32 and two antenna 34, 36. The constructional details of the second embodiment 24 are substantially identical to those described with respect to the first embodiment 10. In particular, an earth plane (not shown) is formed on the face of the substrate 26 opposite the face shown in FIG. 2. By providing two sensors 28, 30 a differential measurement system can be provided, for example to measure differences in pressure. The second embodiment 24 shown in FIG. 2 could be mounted, for example, on a vascular graft. Two antennas 34, 36 are provided and could be used, for example, for separate broadcasting. It is also possible to provide an embodiment having two sensors 28, 30 and a single antenna.

FIG. 3 shows a third embodiment of a device of the invention, depicted generally at 38. The device comprises a substrate 40 in which a portion of the substrate in the region 42 acts as a piezoelectric sensor owing to an electrode formed thereon. The device 38 further comprises integrated circuit 44 and antenna 46. An earth plane (not shown) is formed on the opposite face of the substrate 40 to that shown in FIG. 3. The constructional details and operation of the device 38 are similar to those of device 10. The device 38 is in the form of a relatively long, thin rectangular strip of relatively high aspect ratio. This configuration is convenient for use in the downstream portion of a heart valve. The device 38 may be used for pressure and/or acoustic signal measurement.

FIG. 4 shows a fourth embodiment of a device of the invention, depicted generally at 48. The device 48 is somewhat similar to the second embodiment shown in FIG. 2, comprising a substrate 50, two sensors 52, 54, an integrated circuit 56, and two antennas 58, 60. The principal difference between the fourth embodiment and second embodiment is that the fourth embodiment is in the form of a relatively long, thin rectangle of relatively high aspect ratio. This configuration is convenient for mounting on a vascular stent. The provision of two sensors 52, 54 permits differential measurements, such as differential pressure measurements to be made.

FIG. 5 shows an alternative approach to the measurement of differential signals in which two devices of the invention 62, 64 are utilised. This approach is particularly advantageous in the measurement of differential pressure and/or acoustic signals across a heart valve. Valve leaflets 66 are depicted in schematic fashion in FIG. 5. It can be seen that the devices 62 and 64 are positioned either side of the valve leaflets 66, enabling the function of the valve leaf at 66 to be assessed. FIG. 6 shows the placement of the devices 62, 64 with respect to a heart valve 68. The position of the leaflets of the heart valve 68 are approximately at the level of the dotted line shown in FIG. 6. As a result of this, there is not a great deal of room to position the device 62, and hence relatively thin devices, such as the third embodiment, are preferred. There is less constraint in the positioning of the device 64, and thus devices having rather lower aspect ratios, such as the first embodiment, may be used. However, it should be noted that in both instances the available volume for accommodation of the devices is limited, and that it is highly advantageous that relatively planar devices (particularly devices which can deform to some extent from planarity) are employed. It is an advantage of the present invention that devices of this type are readily provided. There are numerous ways in which the devices 62, 64 might be positioned with respect to the heart valve 68. For example, a pericardial cover might be provided exterior to the main valve wall, and the devices 62, 64 might be disposed between the pericardial cover and the main valve wall.

FIG. 8 shows a fifth embodiment of a medical device, comprising an arrangement for mounting an RFID integrated circuit 90 in flip-chip form onto four contact pads 92, 94, 96, 98. The RFID 90 is mounted on a substrate 100 formed from a suitable material, such as Mylar or another flexible substance. An antenna coil 102 is also disposed on the substrate 100. The antenna coil is of a spiral shape although for reasons of presentational simplicity, a circular shape is shown in FIG. 8. Two of the contact pads 92, 94 are used to make electrical connection to the antenna coil 102, whilst the other pair of contact pads 96, 98 are used to bring electrical connections through to two further contact pads (not shown) which are disposed on the rear face of the substrate 100. The contact pads disposed on the rear face of the substrate 100 are used to make electrical connection with contact pads 104, 106 which are disposed on a front face of a PVDF substrate 108. Disposed over a substantial portion of the front face of the PVDF substrate 108 is an electrode 110 which is configured so that it does not quite reach the edges of the PVDF substrate. The contact pad 104 makes electrical connection with the electrode 110, whilst the contact pad 106 has a plated through hole which enables electrical connection to an earth plane (not shown) which covers the complete rear surface of the PVDF substrate 108. To fabricate the device, the two substrates 100, 108 are brought together into intimate contact. This can be done using a non-conducting adhesive, such as an epoxy adhesive except in the areas of the contact pads required to make electrical connection, in which areas small amounts of a conducting adhesive might be employed. Suitable examples include a silver loaded epoxy adhesive or an anisotropic conducting adhesive employing hollow gold plated polymer spheres, typically of 2 to 3 μm diameter. The bonding arrangement to complete the device could be conducted under vacuum to exclude air pockets between the two substrates. The advantages associated with this form of device construction are principally two-fold: firstly, the PVDF sensor occupies a substantial portion of the device, giving rise to improved sensitivity; and secondly the antenna coil can be made as large as possible for a given substrate dimension, thus improving coupling between the device and an external reader. The advantages discussed previously with respect to the provision of an earth plane also apply. The device is particularly useful for making pressure and/or acoustic measurements.

FIG. 9 shows a sixth embodiment of a medical device comprising a PVDF substrate 112 on which is formed a metallised antenna/electrode 114. An integrated circuit 116, which is preferably an RFID, is disposed on the substrate 112 and is in communication with the antenna/electrode 114. In this embodiment, a single structure 114 acts as both the antenna and the PVDF electrode. Standard methods of polarising the PVDF can be employed. It is advantageous that the antenna/electrode 114 should cover as much of the active area of the PVDF substrate 112 as possible. This can be done by ensuring that the gap between the spiral track making up the antenna/electrode 114 is minimised. It is possible to achieve gaps of about 10 μm using standard methods of metallic deposition. Preferably, the device further comprises a ground electrode or earth plane covering the entire rear surface (not shown) of the PVDF substrate 112. The earth plane could be connected to the integrated circuit 116 via a plated through hole. The device is particularly useful for making acoustic and/or pressure measurements, although different measurements might be contemplated, for example using different materials. If differential measurements, e.g. of pressure, are required then it is possible to provide devices in which a single integrated circuit addresses two separate antenna. Alternatively, two completely separate substrates might be employed each having its own integrated circuit. It is preferred that the substrate is made from the material that acts as the active sensing material in the sensor. However, the principle of utilising a single electrode to poll/power a sensor and to act as an antenna can be applied more generally, e.g. to devices where a separate substrate is used.

FIG. 10 shows in schematic form a system in which differential measurements can be made in vivo. In FIG. 10, the outputs of four sensors 118, 120, 122, 124 are input into a differential measurement device 126 which provides an output related to differences in the responses of the sensors. The output of the differential measurement device 126 is transmitted to telemetric communication means, which preferably comprises an integrated circuit 128 such as an RFID and an antenna 130. The differential measurement device 126 might be a Wheatstone bridge, or any other suitable bridge arrangement. The sensors 118, 120, 122, 124 might comprise top electrodes of a PVDF device of the type described previously which have been partitioned into four separate regions. In this way, greater immunity is achieved from interferences such as electromagnetic noise. Alternatively, the outputs of fewer sensors might be connected to a Wheatstone bridge or other bridge arrangement. Alternatively still, the outputs of e.g. two sensors might be connected to a differential charge amplifier. In this way, the device could switch between the measurement of differential values to the measurement of absolute sensor values. Such an arrangement is particularly useful for making differential measurements of pressure, and a device of the type described with regard to FIG. 9 in which two antenna/electrodes are provided is particularly preferred.

Further improvements are possible if an earth plane is used in conjunction with the remote, reader device as well as the implant device. FIG. 7 shows in schematic form the arrangement contemplated, which comprises an implanted device 70 and a remote, reader device 72. The implanted device 70 comprises an antenna 74 backed by an earth plane 76. Similarly, the reader device 72 comprises an antenna 78 backed by an earth plane 80. It will be appreciated that, for presentation of simplicity, other elements of the implanted device 70 and remote, reader device 72 have been omitted. It should be noted that the antenna and earth plane will generally be disposed on opposite faces of a substrate in the case of both the implanted device 70 and the remote reader device 72. However, it is not necessary that, in the case of the implanted device 70, the substrate is formed from the same material that the sensor is made from. Typically, the separation between reader antenna and earth plane is around 1 to 2 millimetres. The arrangement shown generally in FIG. 7 provides improvements in the telemetric communication to and from the implanted device 70. Without wishing to be bound by any particular theory, it is believed that the improvements are due to improved magnetic field coupling. More efficient telemetric communication is achieved, i.e. for a fixed level of transmitted energy from the implanted device 70, it is possible to penetrate through thicker biological tissue when earth planes are applied to both the implanted device and the remote, reader system.

1. A medical device for implantation in a body including:
a substrate formed from a material capable of acting as an active sensing material for sensing a physiologically or clinically relevant parameter;
at least one sensor for sensing a physiologically or clinically relevant parameter, each sensor including a portion of the substrate which is configured to act as an active sensing material; and
telemetric communication means coupled to said at least one sensor for telemetrically transmitting data related to a parameter sensed by the at least one sensor to a remote device.

2. A medical device according to claim 1 in which the telemetric communication means includes at least one antenna.

3. A medical device according to claim 2 in which the telemetric communication is disposed, at least in part, on the substrate.

4. A medical device according to claim 3 in which the antenna is disposed on the substrate.

5. A medical device according to claim 2 further including an additional substrate having the antenna formed thereon, the additional substrate contacting the substrate formed from a material capable of acting as an active sensing material.

6. A medical device according to claim 1, in which the substrate is substantially planar or bent from a substantially planar configuration.

7. A medical device according to claim 1, in which one face of the substrate has an earth plane disposed thereon.

8. A medical device according to claim 7 in which the telemetric communication means includes at least one antenna, the at least one sensor is disposed on a front face of the substrate, and the earth plane is disposed on a back face of the substrate so as to extend at least over a region which is in register with the area defined by the at least one antenna, and preferably, to extend additionally over a region which is in register with the area defined by a sensor.

9. A medical device according to claim 1, in which the at least one sensor is a piezoelectric sensor.

10. A medical device according to claim 9 in which the substrate is formed from a polymeric material capable of acting as an active piezoelectric sensing material.

11. A medical device according to claim 10 in which the polymeric material comprises PVDF.

12. A medical device according to claim 1, in which the at least one sensor senses pressure and/or acoustic signals.

13. A medical device according to claim 1, in which the telemetric communication means is a passive device, preferably a passive device which is powered by energy transmitted by a remote device.

14. A medical device according to claim 1, in which the telemetric communication means is a transponder.

15. A medical device according to claim 1, in which the telemetric communication means includes an RFID device.

16. A medical device according to claim 1, in which the telemetric communication means includes an integrated circuit.

17. A medical device according to claim 16 in which the integrated circuit is disposed on the substrate.

18. A medical device according to claim 1, adapted to be implanted in the heart of a patient and operable therein is as a heart valve; or ii) to assist in the functioning of one of the patient's heart valves; or iii) to monitor the functioning of one of the patient's heart valves.

19. A medical device according to claim 18 in which the medical device is a heart valve being a tissue valve device having a valve wall formed from tissue.

20. A medical device according to claim 19 further comprising a stent support for the valve wall, in which the substrate, at least one sensor and telemetric communication means are disposed between the stent support and the valve wall.

21. A stentless medical device according to claim 19.

22. A medical device according to claim 18 further including a protective cover disposed around the periphery of the device, in which the substrate, at least one sensor and telemetric communication means are disposed between the valve wall and the protective cover.

23. A medical device according to claim 18 in which the medical device is a mechanical heart valve.

24. A medical device for implantation in a body including:
a substrate or a substrate stack including a plurality of stacked substrates, the substrate or substrate stack having opposed first and second faces;
at least one sensor disposed on the substrate or substrate stack for sensing a physiologically or clinically relevant parameter;
telemetric communication means, coupled to the at least one sensor, for telemetrically transmitting data related to a parameter sensed by the at least one sensor to a remote device, the telemetric communication means including at least one antenna disposed on the first face of the substrate or substrate stack; and
an earth plane disposed on the second face of the substrate or substrate stack and extending at least over a region which is in register with the area defined by the at least one antenna.

25. A system for monitoring a patient including:
a medical device according to claim 24; and
a remote device for receiving data telemetrically transmitted by the telemetric communication means;
in which the remote device includes at least one data receiving antenna and an earth plane extending at least over a region which is in register with the area defined by the at least one data receiving antenna so as to improve the reception of data transmitted telemetrically by the medical device.

26. A sensor including a material capable of acting as an active sensing material and an electrode formed on the material in an antenna pattern so as to i) enable the material to be operated as an active sensing material and ii) act as an antenna to transmit and/or receive signals relevant to the operation of the sensor.

27. A sensor according to claim 26 in which the material is a material capable of acting as an active piezoelectric material.

28. A sensor according to claim 27 in which the material is a polymeric material, preferably PVDF.

29. A sensor according to claim 26 in which the antenna pattern leaves one or more gaps between adjacent portions of the electrode, and the gaps are of less than 50 μm, preferably less than 25 μm, most preferably about 10 μm.

30. A sensor according to claim 26 further including an earth plane formed on the material and extending at least over a region of the material which is in register with the area defined by the electrode.

31. A sensor according to claim 26 in the form of a medical device suitable for implantation in a body, in which the sensor is for sensing a physiologically or clinically relevant parameter, and the electrode telemetrically transmits data related to a parameter sensed by the sensor to a remote device.

32. A medical device for implantation in a body including:
at least two sensors for sensing a physiologically or clinically relevant parameter;
differential measurement means for measuring differences in the responses of the sensor; and
telemetric communication means coupled to the differential measurement means for telemetrically transmitting data related to the differences in the responses of the sensors to a remote device.

33. A medical device according to claim 32 in which the differential measurement means includes a bridge arrangement.

34. A medical device according to claim 33 in which the differential measurement means includes a Wheatstone bridge.

35. A medical device according to claim 32 in which the differential measurement means includes a differential amplifier.

36. (canceled)
